SEQUENCE LISTING (4 sequences)

SEQ ID NO: 1 - DNA, 2649 bp, Rattus norvegicus
atgcctgccctggcgtgcctccggaggctgtgtcggcacctgtccccacaggctgtcctgttcctgctgtttgtcttctgcctgttcagcgtgtttgtctcggcctactacctatatggttggaaccggggcctcgagccctcggcagatgcttctgagtccgactgcggggacccaccacctgtcgcccctagccgtctcctgccaatcaagcctgtgcaggcggtcgccccttctcgaacagacccgctggtgctggtatttgtggagagcctctattcacagctgggccaggaggtggtggccatcctggaatccagtcgcttcaagtaccgaacagaaattgcaccggggaagggggacatgcccacactcacagacaagggccgaggccgcttcgccctcatcatctatgagaacatcctcaagtatgtcaacctggatgcctggaaccgggagctgctggacaagtactgtgtggcctacggcgtgggcatcattggcttcttcaaggccaatgagaacagcctgctgagtgcacagctcaaaggcttccctcttttcctgcattcgaacctgggcttgaaagactgcagcatcaaccccaagtccccactgctgtacgtgacacggcccagtgaggtagagaaaggtgtgctgcccggagaggactggacggtgttccagtctaaccactctacctatgagccagtgctgctggccaagacgcgctcctctgagtccatcccacacctgggcgcagatgccggcctgcatgctgccctgcacgctactgtggtccaggacctgggcctccatgacggcattcagcgtgtgctgtttggcaacaacctcaacttttggctgcataagctcgtcttcgtggacgctgtggccttcctcacagggaagcgcctctcactgcctttggaccgatacatcctggtggacattgatgacatttttgtaggcaaggagggcacacgcatgaaggtggaggatgtgaaggccctgtttgatacacagaatgaacttcgtacacatatcccaaacttcaccttcaacctgggctactcagggaaattcttccacacaggtaccgatgctgaggatgctggggacgacctgctgctgtcctatgtgaaagagttctggtggttcccccacatgtggagccatatgcaaccccacctcttccacaaccagtctgtgctggctgagcagatggccctgaacaagaagttcgctgtcgagcacggcattcccacagatatggggtatgcagtggcaccccaccactctggtgtgtaccctgtgcatgtgcagctgtatgaggcctggaagcaagtgtggaacatccgtgtgaccagcacagaggagtacccgcatctgaagcctgcccgttaccgccgtggcttcatccacaatggcatcatggtcctccctcggcagacctgtggtctctttacacacaccatcttctacaacgagtaccctggaggctccagtgagctggacaagatcatcaatgggggcgagctctttcttactgtgctcctcaatcctatcagcgtcttcatgacacacttatccaactatggaaatgaccgcctgggactgtacaccttcaagcacctggtgcgcttcctgcactcctggaccaacctgaggctgcagacgctgccccctgtgcagctggcccagaagtacttccagatcttttctgaggagaaggacccactttggcaggatccctgtgaggacaaacgccacaaagacatctggtctaaggagaagacatgtgatcgcttcccaaagctgctcatcattggcccccagaaaacaggcaccacagccctctacctgttcctgggcatgcaccccgacctcagcagcaactaccccagctccgagacctttgaggagatccagttttttaatggccacaactatcacaaaggcatcgactggtacatggaattcttccctattccctccaacaccacctctgacttctactttgaaaaaagtgccaactactttgattcagaagtggcaccacggcgagcagctgccctattgcccaaggccaaggttctcaccatcctcatcaatccagccgaccgggcttactcctggtaccagcaccagcgggcccatgatgacccggtggccctaaagtacaccttccatgaggtgatcacagctggccctgacgcatcctcaaagctgcgtgccctccagaaccgatgcctggtccccggctggtatgccactcatattgaacgctggctcagcgcctttcatgccaaccagatcctggtcttggatggcaaactgctgcgaacagaacctgccaaagtgatggacacagtgcagaaattcctcggggtgaccagcacggttgactaccataaaaccttggcgtttgacccaaagaaaggattttggtgccagctgctcgaaggaggaaaaaccaagtgtctgggaaaaagcaagggacggaaatatccagagatggacctggattcccgagccttcctaaaggattactaccgggaccacaacattgagctctctaagctgctgtataagatgggccagacactgcccacctggctgcgggaagacctccagaacaccaggtag

SEQ ID NO: 2 - protein, 882 aa, Rattus norvegicus
MPALACLRRLCRHLSPQAVLFLLFVFCLFSVFVSAYYLYGWNRGLEPSADASESDCGDPPPVAPSRLLPIKPVQAVAPSRTDPLVLVFVESLYSQLGQEVVAILESSRFKYRTEIAPGKGDMPTLTDKGRGRFALIIYENILKYVNLDAWNRELLDKYCVAYGVGIIGFFKANENSLLSAQLKGFPLFLHSNLGLKDCSINPKSPLLYVTRPSEVEKGVLPGEDWTVFQSNHSTYEPVLLAKTRSSESIPHLGADAGLHAALHATVVQDLGLHDGIQRVLFGNNLNFWLHKLVFVDAVAFLTGKRLSLPLDRYILVDIDDIFVGKEGTRMKVEDVKALFDTQNELRTHIPNFTFNLGYSGKFFHTGTDAEDAGDDLLLSYVKEFWWFPHMWSHMQPHLFHNQSVLAEQMALNKKFAVEHGIPTDMGYAVAPHHSGVYPVHVQLYEAWKQVWNIRVTSTEEYPHLKPARYRRGFIHNGIMVLPRQTCGLFTHTIFYNEYPGGSSELDKIINGGELFLTVLLNPISVFMTHLSNYGNDRLGLYTFKHLVRFLHSWTNLRLQTLPPVQLAQKYFQIFSEEKDPLWQDPCEDKRHKDIWSKEKTCDRFPKLLIIGPQKTGTTALYLFLGMHPDLSSNYPSSETFEEIQFFNGHNYHKGIDWYMEFFPIPSNTTSDFYFEKSANYFDSEVAPRRAAALLPKAKVLTILINPADRAYSWYQHQRAHDDPVALKYTFHEVITAGPDASSKLRALQNRCLVPGWYATHIERWLSAFHANQILVLDGKLLRTEPAKVMDTVQKFLGVTSTVDYHKTLAFDPKKGFWCQLLEGGKTKCLGKSKGRKYPEMDLDSRAFLKDYYRDHNIELSKLLYKMGQTLPTWLREDLQNTR

SEQ ID NO: 3 - DNA, 2517 bp, Rattus norvegicus
ctcgagccctcggcagatgcttctgagtccgactgcggggacccaccacctgtcgcccctagccgtctcctgccaatcaagcctgtgcaggcggtcgccccttctcgaacagacccgctggtgctggtatttgtggagagcctctattcacagctgggccaggaggtggtggccatcctggaatccagtcgcttcaagtaccgaacagaaattgcaccggggaagggggacatgcccacactcacagacaagggccgaggccgcttcgccctcatcatctatgagaacatcctcaagtatgtcaacctggatgcctggaaccgggagctgctggacaagtactgtgtggcctacggcgtgggcatcattggcttcttcaaggccaatgagaacagcctgctgagtgcacagctcaaaggcttccctcttttcctgcattcgaacctgggcttgaaagactgcagcatcaaccccaagtccccactgctgtacgtgacacggcccagtgaggtagagaaaggtgtgctgcccggagaggactggacggtgttccagtctaaccactctacctatgagccagtgctgctggccaagacgcgctcctctgagtccatcccacacctgggcgcagatgccggcctgcatgctgccctgcacgctactgtggtccaggacctgggcctccatgacggcattcagcgtgtgctgtttggcaacaacctcaacttttggctgcataagctcgtcttcgtggacgctgtggccttcctcacagggaagcgcctctcactgcctttggaccgatacatcctggtggacattgatgacatttttgtaggcaaggagggcacacgcatgaaggtggaggatgtgaaggccctgtttgatacacagaatgaacttcgtacacatatcccaaacttcaccttcaacctgggctactcagggaaattcttccacacaggtaccgatgctgaggatgctggggacgacctgctgctgtcctatgtgaaagagttctggtggttcccccacatgtggagccatatgcaaccccacctcttccacaaccagtctgtgctggctgagcagatggccctgaacaagaagttcgctgtcgagcacggcattcccacagatatggggtatgcagtggcaccccaccactctggtgtgtaccctgtgcatgtgcagctgtatgaggcctggaagcaagtgtggaacatccgtgtgaccagcacagaggagtacccgcatctgaagcctgcccgttaccgccgtggcttcatccacaatggcatcatggtcctccctcggcagacctgtggtctctttacacacaccatcttctacaacgagtaccctggaggctccagtgagctggacaagatcatcaatgggggcgagctctttcttactgtgctcctcaatcctatcagcgtcttcatgacacacttatccaactatggaaatgaccgcctgggactgtacaccttcaagcacctggtgcgcttcctgcactcctggaccaacctgaggctgcagacgctgccccctgtgcagctggcccagaagtacttccagatcttttctgaggagaaggacccactttggcaggatccctgtgaggacaaacgccacaaagacatctggtctaaggagaagacatgtgatcgcttcccaaagctgctcatcattggcccccagaaaacaggcaccacagccctctacctgttcctgggcatgcaccccgacctcagcagcaactaccccagctccgagacctttgaggagatccagttttttaatggccacaactatcacaaaggcatcgactggtacatggaattcttccctattccctccaacaccacctctgacttctactttgaaaaaagtgccaactactttgattcagaagtggcaccacggcgagcagctgccctattgcccaaggccaaggttctcaccatcctcatcaatccagccgaccgggcttactcctggtaccagcaccagcgggcccatgatgacccggtggccctaaagtacaccttccatgaggtgatcacagctggccctgacgcatcctcaaagctgcgtgccctccagaaccgatgcctggtccccggctggtatgccactcatattgaacgctggctcagcgcctttcatgccaaccagatcctggtcttggatggcaaactgctgcgaacagaacctgccaaagtgatggacacagtgcagaaattcctcggggtgaccagcacggttgactaccataaaaccttggcgtttgacccaaagaaaggattttggtgccagctgctcgaaggaggaaaaaccaagtgtctgggaaaaagcaagggacggaaatatccagagatggacctggattcccgagccttcctaaaggattactaccgggaccacaacattgagctctctaagctgctgtataagatgggccagacactgcccacctggctgcgggaagacctccagaacaccaggtag

SEQ ID NO: 4 - protein, 838 aa, Rattus norvegicus
LEPSADASESDCGDPPPVAPSRLLPIKPVQAVAPSRTDPLVLVFVESLYSQLGQEVVAILESSRFKYRTEIAPGKGDMPTLTDKGRGRFALIIYENILKYVNLDAWNRELLDKYCVAYGVGIIGFFKANENSLLSAQLKGFPLFLHSNLGLKDCSINPKSPLLYVTRPSEVEKGVLPGEDWTVFQSNHSTYEPVLLAKTRSSESIPHLGADAGLHAALHATVVQDLGLHDGIQRVLFGNNLNFWLHKLVFVDAVAFLTGKRLSLPLDRYILVDIDDIFVGKEGTRMKVEDVKALFDTQNELRTHIPNFTFNLGYSGKFFHTGTDAEDAGDDLLLSYVKEFWWFPHMWSHMQPHLFHNQSVLAEQMALNKKFAVEHGIPTDMGYAVAPHHSGVYPVHVQLYEAWKQVWNIRVTSTEEYPHLKPARYRRGFIHNGIMVLPRQTCGLFTHTIFYNEYPGGSSELDKIINGGELFLTVLLNPISVFMTHLSNYGNDRLGLYTFKHLVRFLHSWTNLRLQTLPPVQLAQKYFQIFSEEKDPLWQDPCEDKRHKDIWSKEKTCDRFPKLLIIGPQKTGTTALYLFLGMHPDLSSNYPSSETFEEIQFFNGHNYHKGIDWYMEFFPIPSNTTSDFYFEKSANYFDSEVAPRRAAALLPKAKVLTILINPADRAYSWYQHQRAHDDPVALKYTFHEVITAGPDASSKLRALQNRCLVPGWYATHIERWLSAFHANQILVLDGKLLRTEPAKVMDTVQKFLGVTSTVDYHKTLAFDPKKGFWCQLLEGGKTKCLGKSKGRKYPEMDLDSRAFLKDYYRDHNIELSKLLYKMGQTLPTWLREDLQNTR

(19) United States
(12) Patent Application Publication
(10) Pub. No.: US 2004/0043447 A1
(43) Pub. Date: Mar. 4, 2004

(54) Production of sulfated polysaccharides using glycosaminoglycan-specific sulfotransferases

(76) Inventors: Sami Saribas, Philadelphia, PA (US); Roger Barthelson, Doylestown, PA (US); Ali Mobessari, Voorhees, NJ (US)

Correspondence Address: MORGAN, LEWIS & BOCKIUS LLP, 1701 MARKET STREET, PHILADELPHIA, PA, 19103-2921, US

(21) Appl. No.: 10/177,687

(22) Filed: Jun. 21, 2002

(51) Int. Cl. C07H 21/04; C08B 37/00; C12P 19/28; C12N 9/10; C12N 9/80; C12P 21/02; C12N 5/06

(52) U.S. Cl. 435/69.1; 435/85; 435/193; 435/228; 435/320.1; 435/325; 536/23.2; 536/54

(57) ABSTRACT

The present invention provides materials and methods for the production of bifunctional enzymes that catalyze the N-deacetylation and N-sulfation of saccharides. The invention also provides conditions and methods for assaying the activity of such enzymes.

### BACKGROUND OF THE INVENTION

Sulfated polysaccharides play a central role in many biological processes, ranging from blood coagulation to intercellular communications (Esko and Lindahl, 2001, J. Clin. Invest. 108(2):169-73; Bernfield et al., 1999, Annu. Rev. Biochem. 68:7729-777) to cancer (Liu et al., 2002, PNAS, 99:568-573). Heparin, for example, is a an unbranched polysaccharide chain of repeating disaccharide units. Heparin is commonly found in connective tissue mast cells and is known to act as an anti-coagulant. Heparin mediates anti-coagulant activity through interaction with antithrombin III, which in turn inhibits the action of thrombin and factor Xa.

The ability of heparin to catalyze antithrombin III binding with thrombin, for example, is based on the nature and the extent of sulfation of the heparin saccharide chain. In the pentasaccharide sequence of heparin that is involved in antithrombin III binding, a 3-O-sulfate moiety on the central D-glucosamine is a primary determinant of the anticoagulant activity of heparin. Removal of sulfate groups from any of the residues in the pentasaccharide sequence diminishes the anticoagulant activity of heparin. Moreover, the heterogeneity of N-sulfation in the production of heparin will have similar moderating effects on its biological activity.

Sulfation of polysaccharides (and other carbohydrates) is catalyzed by a group of enzymes known as sulfotransferases. Specifically, type II membrane bound sulfotransferases are Golgi enzymes which utilize the biological high energy sulfate donor PAPS (adenosine 3′phosphate phosphosulfate) to transfer a sulfate group to a specific position on variety of carbohydrate residues (See recent reviews, Fukuda et al., 2001, JBC, 276:47747-47750; Esko and Lindahl, 2001, J. Clin. Invest. 108(2):169-73; Forsberg and Kjellen, 2001, J. Clin. Invest. 108(2):175-80).

Heparan Sulfate/Heparin N-deacetylase/N-sulfotransferase (NDST) is one example of a sulfotransferase enzyme. The synthesis of heparan sulfate begins with a sugar building block consisting of N-acetyl glucosamine (GlcNAc) and glucuronic acid (GlcA). After the formation of a repeating GlcNAc-GlcA backbone, acetyl groups are removed from the GlcNAc residues, and the newly formed free amines on the GlcN residues are sulfated in the presence of PAPS. N-sulfation, the end result of the NDST reaction, plays a critical role in determining the ultimate extent of all sulfation in heparan sulfate chains (Bame and Esko, 1989, J. Biol. Chem. 264(14):8059-65).

The known NDST enzyme possesses both N-deacetylation and N-sulfation activities in a single polypeptide. NDST action on the growing heparan sulfate chain is required for subsequent C-5 epimerization of the GlcA to IdoA by C5-epimerase, the 2-O-sulfation of IdoA by 2-O-Sulfotransferase and 6-O or 3-O sulfation of IdoA by 6-O-sulfotransferase and 3-O sulfotransferase, respectively (Lindahl et al., 1998, J. Biol. Chem. 273:24479-24982; Perrimon and Bemfield, 2000, Nature, 404:725-728).

NDST-encoding genes have been cloned from many different mammalian sources (Hashimoto et al., 1992 J. Biol. Chem. 267:15744-15750; Orellana, et al., 1994, J. Biol. Chem. 269:2270-2276; Kushe-Gullberg et al.,1998, J. Biol. Chem. 273:11902-11907; Toma et al., 1998, J. Biol. Chem. 273:22458-22465). Two forms of the enzyme sharing 70% homology have been identified and are called NDST1 and NDST2. Studies on NDST genes in mice demonstrated that an NDST1 knock-out resulted in neolethality, whereas an NDST2 knock-out resulted in a deficiency in heparin biosynthesis (Humphries et al., 1999, Nature 400:269-772; Forsberg et al., 1999, Nature 400:773-776; Ringwall et al., 2000, J. Biol. Chem. 275:25926-25930). Pikas, et al. (2000, Biochemistry 39:4552-4558) have recently studied the extent of N-sulfation in saccharides and found that the sulfation was higher in an NDST2-catalyzed reaction than in an NDST1-catalyzed reaction.

Although it is believed that NDST1 is primarily responsible for heparan sulfate biosynthesis and NDST2 is primarily responsible for heparin biosynthesis in vivo, Toma et al. (1998, J. Biol. Chem. 273:22458-22465) have shown that NDST2 is also present in non-heparin producing cells. Recently, two new types of the enzyme, NDST3 and NDST4, have been cloned and expressed in Chinese Hamster Ovary (CHO) cells (Aikawa and Esko, 1999, J. Biol. Chem. 274:2690-2695; Aikawa et al., 2001, J. Biol. Chem. 276:5876-5882). All four NDST's have somewhat different N-sulfotransferase and N-deacetylase activities. For example, the highest N-sulfotransferase activity was observed in NDST1, and NDST2 exhibits equal amounts of both activities.

NDST1 is a bifunctional enzyme catalyzing both the deacetylation of a GlcNAc residue in a sugar chain that is composed of GlcNAc-GlcA repeating disaccharide units, such as E. coli K5 polysaccharide, and the subsequent sulfation of the deacetylated amino group to yield an N-sulfated product in GlcNS form. To date, in vitro assays for each activity have been conducted in separate reaction mixtures, using different reaction conditions. In vitro assays for the deacetylase activity are conducted using a specific substrate, such as acetyl-radiolabeled heparosan, after which the reaction mixture is assessed for the presence of liberated radioactive acetyl groups. The sulfotransferase activity of NDST is assessed separately using a different substrate, such as ³⁵S-labeled PAPS, using heparosan or N-desulfated heparin. The degree of sulfotransferase activity is measured by determining the amount of radiolabeled sulfur incorporated into the saccharide chain.

As noted, in addition to the requirement for separate substrates for the deacetylation and sulfation reactions of NDST assays, the reaction conditions also differ. For example, the deacetylase reaction is often conducted at pH 6.5, while the sulfation reaction is conducted at a pH of 7.0.

The NDST isozymes all seem to function to N-acetylate and N-sulfate GlcNAc residues in heparan sulfate/heparin biosynthesis, but each activity operates in different tissues (Aikawa et al., 2001, J. Biol. Chem. 276:5876-5882). Rat liver NDST1 was first cloned and expressed as protein A fusion protein in CHO cells (Hashimoto et al., 1992 J. Biol. Chem. 267:15744-15750). Beminsone and Hirschberg (1998, J. Biol. Chem. 273:25556-25559) demonstrated that the N-sulfotransferase activity of the rat liver enzyme is located in carboxyl half of the protein, and the amino-terminal portion of the enzyme possesses N-deacetylase activity. The N-sulfotransferase region also contains a PAPS binding domain common to all sulfotransferases.

Bacterial expression of sulfotransferases is often difficult. To date, the majority of the carbohydrate sulfotransferases have been expressed in either soluble form or as full-length proteins in mammalian cells, i.e., in either CHO or COS cells. However, successful bacterial expression of a carbohydrate sulfotransferase was recently achieved by cloning the sulfotransferase domain of the human NDST as a glutathione-S-transferase (GST) fusion protein (Sueyoshi et al., 1998, FEBS Letters 433:211-214). Further, heparan sulfate 3-O-sulfotransferase has been expressed in bacteria (Myette et al., 2002, Biochem. Biophys. Res. Comm. 290:1206-1213).

In addition to the in vivo functions of naturally-produced heparin, exogenously-administered heparin has many therapeutic uses. For example, heparin therapy is used in the treatment of thrombosis. However, heparin-induced thrombocytopenia (HIT) is a common adverse side effect of heparin therapy. In one type of HIT, platelet aggregation is believed to occur as a result of the heparin treatment itself. Another form of HIT occurs when heparin-antibody complexes bind to platelets resulting in platelet activation and thrombocytopenia, among other manifestations.

Several explanations have been proposed for the causes of thrombocytopenia. It was found that significantly more thrombocytopenic events occurred when using heparin obtained from bovine sources. A subsequent switch to heparin isolated from porcine sources decreased the number of cases of HIT. More recently, it has been found that lower molecular weight heparins (LMWH), which are actually depolymerization products of heparin, provide a higher ratio of anticoagulant effects of heparin and decrease the incidence of HIT. However, either of the aforementioned variations of heparin therapy results in quite large variations in the therapeutic effects of heparin.

Noting the variability in therapeutic effect with different forms and/or sources of heparin, the FDA often treats a variation of a known heparin as a “new” anticoagulant. However, because of the efficacy of heparin therapy, it is desirable to increase the consistency in heparin preparations.

The importance of proper sulfation of heparin and heparan sulfate is well-documented, and the adverse clinical manifestations arising due to the prevalence of heterogeneous sulfation of heparin and heparan sulfate are well known in the art. Accordingly, there is a long-felt need for a way to produce or refine sulfation patterns of heparin and heparan sulfate in order to minimize or eliminate adverse side effects linked with heparin and heparan sulfate therapy.

### BRIEF SUMMARY OF THE INVENTION

The invention includes a polypeptide that is a bifunctional enzyme having both N-deacetylase and N-sulfotransferase activities, methods of using the polypeptide, an isolated nucleic acid encoding the polypeptide, vectors containing the nucleic acid, and cells containing the vectors.

In one embodiment of the invention, an isolated nucleic acid encoding a bifunctional enzyme having both N-deacetylase and N-sulfotransferase activities, wherein both activities are active in the same in vitro reaction mixture, is provided. Another embodiment of the invention provides the polypeptide encoded by the isolated nucleic acid, referred to herein as rNDST1.

An embodiment of the invention provides an isolated nucleic acid that is a homolog, variant, mutant or fragment of a nucleic acid encoding a bifunctional enzyme having both N-deacetylase and N-sulfotransferase activities, wherein both activities are active in the same in vitro reaction mixture. Another embodiment of the invention provides the polypeptide encoded by the isolated nucleic acid that is a homolog, variant, mutant or fragment of a nucleic acid encoding a bifunctional enzyme having both N-deacetylase and N-sulfotransferase activities.

One embodiment of the invention provides an isolated nucleic acid that is at least 99% identical to a nucleic acid encoding a bifunctional enzyme having both N-deacetylase and N-sulfotransferase activities, wherein both activities are active in the same in vitro reaction mixture. Another embodiment of the invention provides the polypeptide encoded by the isolated nucleic acid that is at least 99% identical to a nucleic acid encoding a bifunctional enzyme having both N-deacetylase and N-sulfotransferase activities.

In an aspect of the invention, the isolated nucleic acid encoding rNDST1 comprises a poly-histidine sequence. In another aspect of the invention, the isolated nucleic acid encoding rNDST1 comprises an Xpress™ epitope. In yet another aspect of the invention, the isolated nucleic acid encoding rNDST1 comprises a enterokinase cleavage site.

In one aspect of the invention, the isolated nucleic acid encoding rNDST1 comprises a poly-histidine sequence, an Xpress™ epitope, and an enterokinase cleavage site. In a further aspect of the invention, a poly-histidine sequence, an Xpress™ epitope, and an enterokinase cleavage site can be removed from the polypeptide encoded by the isolated rNDST1 nucleic acid through enzymatic cleavage catalyzed by an enterokinase.

In one aspect of the invention, a method of N-deacetylating and N-sulfating a saccharide comprises contacting a saccharide with a composition comprising rNDST1, under conditions sufficient to support both activities in the same reaction mixture such that the saccharide is modified by N-deacetylation and N-sulfation reactions catalyzed by rNDST1. In yet another aspect of the invention, the N-deacetylation and N-sulfation reactions are both conducted in the same reaction mixture.

In another aspect of the invention, a method of N-deacetylating and N-sulfating a saccharide comprises contacting a saccharide with a composition comprising a homolog, variant, mutant, or fragment of rNDST1, under conditions sufficient to support both activities in the same reaction mixture such that the saccharide is modified by N-deacetylation and N-sulfation reactions catalyzed by rNDST1. In yet another aspect of the invention, a method of N-deacetylating and N-sulfating a saccharide comprises contacting a saccharide with a composition comprising a polypeptide that is at least 99% identical to rNDST1, under conditions sufficient to support both activities in the same reaction mixture such that the saccharide is modified by N-deacetylation and N-sulfation reactions catalyzed by rNDST1.

In an embodiment of the present invention, the rNDST1 N-deacetylation and N-sulfation reactions are both conducted in the same reaction mixture, wherein the reaction mixture is a cell extract.

In one embodiment of the invention, an isolated nucleic acid encoding rNDST1, wherein both activities are active in the same in vitro reaction mixture, further comprises a nucleic acid specifying a promoter and/or regulatory sequence operably linked to the isolated nucleic acid encoding rNDST 1. In yet a further embodiment of the invention, the promoter is functional in a yeast, fungus, bacterial, insect, or mammalian expression system.

In one aspect of the invention, a vector comprises an isolated nucleic acid encoding rNDST1 or a fragment thereof. In another aspect of the invention, the vector further comprises a nucleic acid specifying a promoter and/or regulatory sequence operably linked to the isolated nucleic acid encoding rNDST1, or fragment thereof. In still another aspect of the invention, the vector comprising a nucleic acid specifying a promoter and/or regulatory sequence operably linked to the isolated nucleic acid encoding rNDST1, or fragment thereof, is expressed when introduced into a cell.

In a further aspect of the invention, a vector containing an isolated nucleic acid encoding rNDST1 also comprises a six-histidine sequence to aid in purification of the expressed polypeptide, an Xpress™ epitope to aid in detection of the polypeptide, or an Enterokinase recognition site for cleavage of the purification and detection sequences from the polypeptide, in an operable linkage to the isolated nucleic acid encoding rNDST1. In yet a further aspect of the invention, two or more sequences operably linked to the isolated nucleic acid encoding rNDST1 may comprise the vector.

In one embodiment of the invention, a recombinant cell comprises an isolated nucleic acid encoding rNDST1, or a fragment thereof. In another embodiment of the invention, a recombinant cell comprising an isolated nucleic acid encoding rNDST1, or a fragment thereof, may be comprised of any vector of the invention.

One aspect of the invention provides a method for detecting sulfotransferase activity of rNDST1 in an assay mixture. A further aspect of the invention provides a method for detecting sulfotransferase activity of rNDST1 in an assay mixture containing specific components, wherein specific assay components comprise rNDST1 enzyme, MnCl₂, MgCl₂, CaCl₂, an acceptor sugar, and both ³⁵S-radiolabeled and non-labeled PAPS.

Another aspect of the invention provides a method for detecting sulfotransferase activity of rNDST1 in an assay mixture, wherein the method comprises a specific assay mixture, a length of time over which the reaction may proceed, the isolation of the sugars from the reaction, and the measurement of radiolabeled sugars in the isolated sugars. In yet a further aspect of the invention, the acceptor sugar in an assay mixture for detection of sulfotransferase activity of rNDST1 is chosen from the group including E. coli K5 polysaccharide, de-N-sulfated heparin, N-desulfated N-acetylated heparin, and completely desulfated N-acetylated heparan sulfate.

In an embodiment of the invention, a method for detecting sulfotransferase activity of rNDST1 in an assay mixture requires a specific buffer system. In a further embodiment of the invention, the specific buffer system has a pH value between 6.5 and 7.0. In still a further embodiment of the invention, the buffer system used in a method for detecting sulfotransferase activity of rNDST1 in an assay mixture includes at least one salt chosen from the group comprising MnCl₂, MgCl₂, CaCl₂. In yet a further aspect of the invention, the buffer system used in a method for detecting sulfotransferase activity of rNDST1 in an assay mixture includes EDTA.

### BRIEF DESCRIPTION OF THE DRAWINGS

FIG. 1a is an image of a gel depicting a single 2.5 bp DNA band corresponding to a truncated NDST gene that was obtained after PCR amplification of the truncated gene from the full-length DNA.

FIG. 1b is a diagram describing the restriction mapping of the pYES2-rNDST1 vector-insert construct. Restriction endonuclease digestion used to map the construct is shown.

FIG. 2 is a graph illustrating the use of a Heparin-Sepharose CL-6B column for partial purification of rNDST1. The rNDST1-containing fractions were identified by assaying eluate fractions for sulfotransferase activity.

FIG. 3 is a graph illustrating the deacetylation of K5 polysaccharide with rNDST1 of the invention. Percent values of K5 deacetylation were calculated based on the HPLC profile of deacetylation products formed over time by the action of rNDST1 at both 37° C. and at room temperature.

FIG. 4 is a graph depicting the N-sulfation of K5 polysaccharide with rNDST1 as a function of time. Sulfotransferase activity was measured as sulfate transfer to K5 polysaccharide, and this N-sulfotransferase activity was linear between 20 and 90 minutes.

FIG. 5a is a graph illustrating an HPLC profile of N-deacetylated E. coli K5 polysaccharides, where the N-deacetylation was carried out at 35° C. overnight with 10 μl rNDST1 enzyme (specific activity 231 pmol/min/mg). Uronic acid/glucosamine (deacetylated product) comprised 30% of the sample and eluted at 0.35 minutes.

FIG. 5b is a graph illustrating an HPLC profile of N-deacetylated P. multicoda polysaccharides, where the N-deacetylation was carried out at 35° C. overnight with 10 μl rNDST1 enzyme (specific activity 231 pmol/min/mg). Uronic acid/glucosamine (deacetylated product) comprised 31% of the sample and eluted at 0.35 minutes.

FIG. 6a is a graph illustrating an HPLC profile of N-sulfated E. coli K5 polysaccharides, where the N-sulfation was carried out at 35° C. overnight with 400 μM PAPS and 5 μl rNDST1 (specific activity 231 pmol/min/mg). N-sulfated uronic acid/N-sulfo-glucosamine comprised 60% of the product, and eluted at 16.3 minutes.

FIG. 6b is a graph illustrating an HPLC profile of N-sulfated P. multicoda polysaccharides, where the N-sulfation was carried out at 35° C. overnight with 400 μM PAPS and 5 μl rNDST1 (specific activity 231 pmol/min/mg). N-sulfated uronic acid/N-sulfo-glucosamine comprised 65% of the product, and eluted at 16.3 minutes.

### DETAILED DESCRIPTION OF THE INVENTION

The present invention includes nucleic acids, proteins, and methods for the production of N-deacetylase/N-sulfotransferase enzymes and the use of these enzymes in production of heparinoids. A key feature of the invention therefore is to design, express and isolate N-deacetylase/N-sulfotransferase enzymes that can synthesize heparin and other similar oligo- and polysaccharides.

The importance of heparin and heparin-like molecules of proper size and with the correct modifications is well known in the art, as are the limitations of present in vitro methods for the production of properly modified and appropriate-sized heparin and heparin-related compounds, particularly when the starting products are extensively heterogeneous.

In the present invention, an isolated N-deacetylase/N-sulfotransferase enzyme has been discovered that possesses both N-deacetylase and N-sulfotransferase activities and catalyzes both N-deacetylase and N-sulfotransferase activities in the same reaction mixture. The enzyme catalyzes the synthesis and modification of heparin, heparin precursors, or heparin-related polysaccharides. The enzyme is produced simultaneously by a cell containing a vector encoding the isolated gene for the N-deacetylase/N-sulfotransferase enzyme.

In one aspect of the invention, there is provided an isolated nucleic acid encoding an N-deacetylase/N-sulfotransferase enzyme capable of catalyzing both N-deacetylation and N-sulfotransfer in the same reaction mixture.

The isolated nucleic acid of the present invention may be isolated from numerous sources, including mammalian tissue, insects, nematodes, and cDNA libraries. The isolated nucleic acid may be characterized using any technique well-known in the art, such as nucleotide sequencing (Sambrook et al., 1989, Molecular Cloning: A Laboratory Manual, Cold Spring Harbor Laboratory, New York). Upon identification of the isolated nucleic acid as encoding a polypeptide having the biological activity of catalyzing both N-deacetylation and N-sulfotransfer reactions in the same reaction mixture, the isolated nucleic acid may be modified as described herein.

The nucleic acid of the invention is exemplified by SEQ ID NO:1, which comprises full-length rat liver NDST cDNA. The corresponding protein is set forth in SEQ ID NO:2. The present invention includes a nucleic acid encoding a truncated form of NDST. In one aspect of the invention, 131 base pairs (bp) are truncated from the 5′ end of the rat liver NDST gene to yield the DNA sequence of SEQ ID NO:3. Deletion of this terminal portion of the gene eliminates from the amino-terminal region of the NDST polypeptide a 42 amino acid sequence comprising the membrane-binding region of the enzyme. SEQ ID NO:4 is the sequence of the encoded polypeptide. As described more fully elsewhere herein, this NDST polypeptide is significantly more soluble than full-length NDST polypeptide.

The invention should not be construed to be limited solely to rat liver NDST, but rather, should be construed to encompass any NDST enzyme, either known or unknown, which is capable of catalyzing in the same reaction, the N-deacetylation and N-sulfation encoding sequences. Modified gene sequences, i.e. genes having sequences that differ from the gene sequences encoding the naturally-occurring proteins, are also encompassed by the invention, so long as the modified gene still encodes a protein having the biological activity of catalyzing both N-deacetylation and N-sulfotransfer reactions in the same reaction mixture. These modified gene sequences include modifications caused by point mutations, modifications due to the degeneracy of the genetic code or naturally occurring allelic variants, and further modifications that have been introduced by genetic engineering, i.e., by the hand of man. Thus, the term nucleic acid also specifically includes nucleic acids composed of bases other than the five biologically occurring bases (adenine, guanine, thymine, cytosine and uracil).

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator <<http://www.ncbi.nlm.nih.gov/BLAST/>>. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See <<http://www.ncbi.nlm.nih.gov>>.

In another aspect of the present invention, a nucleic acid encoding an N-deacetylase/N-sulfotransferase enzyme may have at least one nucleotide inserted into the naturally-occurring nucleic acid sequence. Alternatively, an additional N-deacetylase/N-sulfotransferase enzyme may have at least one nucleotide deleted from the naturally-occurring nucleic acid sequence. Further, an N-deacetylase/N-sulfotransferase enzyme of the invention may have both a nucleotide insertion and a nucleotide deletion present in a single nucleic acid sequence encoding the enzyme.

Techniques for introducing changes in nucleotide sequences that are designed to alter the fimctional properties of the encoded proteins or polypeptides are well known in the art. Such modifications include the deletion, insertion, or substitution of bases, and thus, changes in the amino acid sequence. As is known to one of skill in the art, nucleic acid insertions and/or deletions may be designed into the gene for numerous reasons, including, but not limited to modification of nucleic acid stability, modification of nucleic acid expression levels, modification of expressed polypeptide stability or half-life, modification of expressed polypeptide activity, modification of expressed polypeptide properties and characteristics, and changes in glycosylation pattern. All such modifications to the nucleotide sequences encoding such proteins are encompassed by this invention.

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

An isolated polynucleotide of the present invention may be cloned into a DNA vector.

In one preferred embodiment, rat liver NDST1 is cloned into an expression vector downstream of the 3′ end of a sequence encoding multiple functional tags. The 5′ end fusion to rNDST1 comprises a six-histidine sequence to aid in purification of the expressed polypeptide, an Xpress™ epitope to aid in detection of the polypeptide, and an Enterokinase recognition site for cleavage of the purification and detection sequences from the polypeptide.

In yet another aspect of the present invention, rNDST1 is expressed in yeast cells, using an appropriate expression vector and yeast cell. However, as evidenced by the literature relevant to the art, one skilled in the art will appreciate that rNDST1 can also be expressed in other eukaryotic cells, including mammalian, insect, or prokaryotic cells, including bacteria. rNDST1 protein of the present invention may be expressed using any technique well-known in the art, such as simple expression, high level expression, or overexpression (Sambrook et al., 1989, Molecular Cloning: A Laboratory Manual, Cold Spring Harbor Laboratory, New York).

In one aspect of the invention, rNDST1 is expressed in Saccharomyces cerevisiae using a vector such as pYES2 or pYD, using a GAL1 promoter and inducing expression with galactose. In yet another aspect of the invention, rNDST1 is expressed in Schizosaccharomyces pombe using a pNMT vector and an nmt1 promoter, inducing expression with thiamine. In still another aspect of the invention, rNDST1 is expressed in Kluyveromyces lactis using a pSPGK1 vector with a GK phosphoglycerate kinase promoter. In another aspect of the invention, rNDST1 is expressed in Pichia pastoris or Pichia methanolica using a pPICZ vector, a promoter such as AOX1 or GAP, and using methanol to induce expression. In a further aspect of the invention, Henseluna polymorpha is used to express rNDST1 from pRBGO vectors using methanol and a formate dehydrogenase promoter. In yet another aspect of the invention, rNDST1 is expressed in Yarrowia lipolytica cells in a LYS2, ARS18, or ARS68 vector, using LEU2 or XPR2 promoters. One skilled in the art would recognize that other expression systems, such as those employing Aspergillus strains, can also be used for expression of rNDST1 (Romanos et al., 1992, Yeast 8:423-488).

The rNDST1 of the present invention may also be expressed in bacterial cells. In one aspect of the invention, rNDST1 is expressed in E. coli using a pBAD vector with an arabinose-induced araBAD promoter. In another aspect of the present invention, rNDST1 is expressed in E. coli using a pET vector with an IPTG-induced T7 promoter.

Insect cells can also be used for expression of rNDST1 of the present invention. In an aspect of the invention, Sf9, Sf21, High Five™ or Drosophila Schneider S2 cells can be used. In another aspect of the invention, a Drosophila expression system can be used with a pMT or pAC5 vector and an MT or Ac5 promoter. In yet another aspect of the invention, baculovirus can be used to express rNDST1 using a pAcGP67, pFastBac, pMelBac, or pIZ vector and a polyhedrin, p10, or OpIE3 actin promoter.

rNDST1 can also be expressed in mammalian cells. In an aspect of the invention, 294, HeLa, Chinese hamster ovary, Jurkat, or COS cells can be used to express rNDST1. For mammalian cell expression of rNDST1, a suitable vector such as pT-Rex, pSecTag2, pBudCE4.1, or pCDNA/His Max vector can be used, along with, for example, a CMV promoter.

The present invention relates to methods utilizing polypeptides encoded by the nucleic acids described above. Such methods may utilize polypeptides of the present invention to catalyze both N-deacetylase and N-sulfotransferase reactions in a single assay. It will be understood that methods in which polypeptides of the present invention catalyze both N-deacetylase and N-sulfotransferase reactions in a single assay will be used under assay conditions sufficient to support both N-deacetylase and N-sulfotransferase reactions simultaneously.

SEQ ID NO:2 illustrates the full-length rat liver NDST1 polypeptide. SEQ ID NO:4 illustrates the truncated form of the NDST1 of the present invention. The truncated form of NDST has a 42 amino acid sequence deleted from the N-terminus of the polypeptide. The 42 amino acid sequence comprises the membrane-binding region of NDST and deletion of this sequence significantly improves the solubility of the polypeptide.

The present invention also provides for analogs of proteins or peptides encoded by N-deacetylase/N-sulfotransferase genes. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In another aspect of the present invention, compositions comprising an isolated N-deacetylase/N-sulfotransferase enzyme may include highly purified N-deacetylase/N-sulfotransferase enzymes. Alternatively, compositions comprising the N-deacetylase/N-sulfotransferase enzymes may include cell lysates prepared from the cells used to express the particular N-deacetylase/N-sulfotransferase enzymes. Further, N-deacetylase/N-sulfotransferase enzymes of the present invention may be expressed in one of any number of cells suitable for expression of polypeptides, such cells being well-known to one of skill in the art. Such cells include, but are not limited to bacteria, yeast, insect, and mammalian cells.

Substantially pure protein isolated and obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

Historically, N-deacetylation and N-sulfotransfer activities were thought to be catalyzed by different enzymes. Until the present invention, those skilled in the art utilized two separate and distinct assays to monitor either the N-deacetylase or the N-sulfotransferase activity of NDST. Despite the knowledge that both activities reside within a single NDST polypeptide, the distinctly different reaction conditions led previous investigators to maintain separate assays to monitor each of the two NDST activities. For example, Aikawa et al. (2001, J. Biol. Chem. 276:5876-5882) assayed the N-sulfotransferase activity of NDST by measuring incorporation of radiolabeled sulfate into heparosan or N-desulfated heparin at pH 7.0 in the presence of magnesium, calcium, and manganese. Alternatively, they assayed the N-deacetylase activity using a separate stock of NDST at pH 6.5 in the presence of manganese (but in the absence of both calcium and magnesium) and with a separately prepared N-acetyl heparosan substrate. The assays were conducted in separate vessels at separate times.

Disclosed herein is a method for the production of soluble rat liver heparin/heparan sulfate N-deacetylase/N-sulfotransferase (rNDST1) enzyme by expressing the isolated gene encoding this enzyme in yeast cells and using the enzyme to manufacture N-sulfated polysaccharides. rNDST1 catalyzes the deacetylation of a GlcNAc residue in a sugar chain that has repeating GlcNAc-GlcA disaccharide units, such as E. coli K5 polysaccharide, and the subsequent sulfation of the deacetylated amino group to yield an N-sulfated product in GlcNS form. The dual activity resides in one enzyme and both activities can be conducted in a single reaction mixture.

The present invention offers a method for sulfating saccharides in a controlled manner. This invention couples the deacetylase and sulfotransferase reactions of rNDST1 in a single reaction mixture. rNDST1 catalyzes both reactions sequentially, sulfating the amino groups exposed by the deacetylation of GlcNAc residues. In order to catalyze this reaction as a “one-reaction mixture” synthesis, reaction conditions are adjusted such that the characteristics of the reaction mixture is sufficiently conducive to both reactions. In a single synthesis reaction, the addition of PAPS to a reaction in progress will initiate the sulfation cycle.

Further, the single reaction synthesis of the present invention uses one substrate. The N-deacetylation reaction of the first part of the synthesis produces the substrate for the second part of the synthesis, the N-sulfation reaction.

In the present invention, heparin and heparan sulfate are N-deacetylated at GlcNAc residues and subsequently N-sulfated at the point of deacetylation. This treatment is carried out in a controlled manner using the rNDST1 enzyme in the presence of 3′-phosphoadenosine-5′-phosphosulfate (PAPS). The activity and characteristics of rNDST1 may be controlled at the nucleic acid level by specific sequence deletions and insertions.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′ end; the left-hand direction of a double-stranded polynucleotide sequence is referred to as the 5′-direction.

A first defined nucleic acid sequence is said to be “immediately adjacent to” a second defined nucleic acid sequence when, for example, the last nucleotide of the first nucleic acid sequence is chemically bonded to the first nucleotide of the second nucleic acid sequence through a phosphodiester bond. Conversely, a first defined nucleic acid sequence is also said to be “immediately adjacent to” a second defined nucleic acid sequence when, for example, the first nucleotide of the first nucleic acid sequence is chemically bonded to the last nucleotide of the second nucleic acid sequence through a phosphodiester bond.

A first defined polypeptide sequence is said to be “immediately adjacent to” a second defined polypeptide sequence when, for example, the last amino acid of the first polypeptide sequence is chemically bonded to the first amino acid of the second polypeptide sequence through a peptide bond. Conversely, a first defined polypeptide sequence is said to be “immediately adjacent to” a second defined polypeptide sequence when, for example, the first amino acid of the first polypeptide sequence is chemically bonded to the last amino acid of the second polypeptide sequence through a peptide bond.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positionss of each of the portions are occupied by the same nucleotide residue. More preferably, all nucleotide residue positions of each of the portions are occupied by the same nucleotide residue.

As used herein, “homology” is used synonymously with “identity.”

The percent identity between two sequences can be determined using techniques similar to those described above, with or without allowing gaps. In calculating percent identity, typically exact matches are counted.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis- acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

An “Enterokinase cleavage site” refers to the amino acid sequence Asp-Asp-Asp-Asp-Lys. This sequence is a recognition sequence for the Enterokinase protease, which will cleave the polypeptide chain immediately after the lysine residue, provided the lysine residue is not followed by a proline residue.

A “bifunctional enzyme” refers to a single polypeptide that possesses two distinguishable catalytic activities. The two enzymatic activities may be functional simultaneously or they may operate only one at a time. Further, the two enzymatic activities may be independent of one another or may exist in a cooperative or synergistic manner.

“N-deacetylation” is the chemical loss or removal of an acetyl functional group from a nitrogen-containing functional group, particularly by way of the cleavage of a bond between the acetyl group and the nitrogen atom of a separate functional group. “N-deacetylase activity” is N-deacetylation as catalyzed by an enzyme.

“N-sulfation” is the chemical addition or bonding of a sulfur-containing functional group from a nitrogen-containing functional group, particularly by way of chemical bond formation between the sulfur-containing group and the nitrogen atom of a separate functional group. “N-sulfotransferase activity” is N-sulfation as catalyzed by an enzyme.

An enzyme having “both N-deacetylase and N-sulfotransferase activity” is a single enzyme capable of catalyzing both N-deacetylation and N-sulfation reactions as described above. The N-deacetylation and N-sulfation reactions may be conducted at the same time in the same reaction mixture, or may be conducted separately in separate reaction mixtures.

The term “saccharide” refers in general to any carbohydrate, a chemical entity with the most basic structure of (CH₂O)ₙ. Saccharides vary in complexity, and may also include nucleic acid, amino acid, or virtually any other chemical moiety existing in biological systems.

“Monosaccharide” refers to a single unit of carbohydrate of a defined identity.

“Oligosaccharide” refers to a molecule consisting of several units of carbohydrates of defined identity. Typically, saccharide sequences between 2-20 units may be referred to as oligosaccharides.

“Polysaccharide” refers to a molecule consisting of many units of carbohydrates of defined identity. However, any saccharide of two or more units may correctly be considered a polysaccharide.

### EXPERIMENTAL EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

Amplification of rNDST1 gene from rat liver cDNA library

One nanogram of rat liver cDNA was amplified with primers, NDASTXH1 (SEQ ID NO:5) and NDASTXB1 (SEQ ID NO:6) using the Herculase High performance polymerase chain reaction (PCR) system (Stratagene, La Jolla, Calif.). The resulting PCR products were separated in 1% Seakem agarose II gel (FMC, Philadelphia, Pa.) and visualized with SyberGreen DNA binding dye (Molecular Probes, Eugene, Ore.). The 2.4 kb PCR product was excised from the agarose gel and purified using Ultrafree DA columns (Millipore, Bedford, Mass.) and subsequently concentrated with YM 100 filters (Millipore, Bedford, Mass.). The DNA fragments were then recovered in 20 μl sterile dH₂O.

Cloning rNDST1 into pCR-Blunt vector

The truncation of rat liver NDST1 gene was performed to eliminate 131 bp of DNA sequence (which encodes 42 amino acids including membrane binding region) from the 5′ end of the gene so that a soluble dual-function enzyme could be created. The truncated form of the gene was amplified by means of PCR and comprises the 2514 bp N-deacetylase and N-sulfotransferase encoding domains (SEQ ID NO:2). The 2514 bp DNA fragment was subsequently cloned into a yeast expression vector, pYES2/NTC (Invitrogen, Carlsbad, Calif.), immediately 3′ downstream from a 147 bp (49 amino acid peptide) region that includes several tags for identification and purification purposes. Expression of the fusion protein produces an amino-terminal truncated recombinant rat liver NDST1 (rNDST1) having a 49 amino acid fusion peptide at the amino-terminal end of the polypeptide. The fusion protein includes a 6-histidine tag, an Xpress™ epitope, and an Enterokinase recognition site. With the addition of the 49 amino acid tag from the pYES2/NTC vector to the 840 amino acid truncated recombinant enzyme, the final fusion protein is 889 amino acids in length.

The purified 2.5 kb PCR product comprising the rNDST1 gene was cloned into Invitrogen's (Carlsbad, Calif.) pCR-Blunt vector. One microliter of PCR-blunt vector was mixed with 2 μl of purified 2.5 kb PCR product containing the rNDST1 gene in the presence of 1×Ligation buffer and 1 μl of T4 ligase. The mixture was incubated at 16° C. for 2 hours in a thermocycler. The ligation mixture was removed from the thermocycler and 2 μl aliquots were mixed with 50 μl of competent E. coli TOP10 cells (Invitrogen, Carlsbad, Calif.). The cells were incubated on ice for 30 minutes and were then subjected to heat shock at 42° C. for 45 seconds. After a 2 minute incubation on ice, 250 μl of prewarmed (at 37° C.) SOC medium was added to the mixture, followed by a 1 hour incubation at 37° C. 10- and 100 μl aliquots were plated on LB+ kanamycin plates (Teknova, Half Moon Bay, Calif.). The plates were incubated in 37° C. incubator overnight. The following day, colonies were obtained on plates (10 and 300 colonies were obtained on the 10- and 100 μl plates, respectively).

Screening for pCR-Blunt-rNDST1 recombinants

Six colonies were selected from the plates and grown in 5 ml of LB supplemented with 75 μg/ml kanamycin. After overnight incubation at 37° C., the cultures were removed from the incubator and plasmids were isolated from the cells using a Miniprep kit (Qiagen, Chatsworth, Calif.). Plasmid DNA was eluted in 40 μl elution buffer (EB; Qiagen, Chatsworth, Calif.). The plasmids were then subjected to restriction enzyme digestion to evaluate and verify the PCR insert. Four μl of plasmid DNA was digested with 20 units each of Xho I and Xba I in 1×NEB 2 restriction buffer (New England Biolabs, Beverly, Mass.) supplemented with BSA (20 μl total volume). The resulting restriction fragments were separated on 1% Seakem agarose gel stained with SyberGreen dye (Molecular Probes, Eugene, Ore.). Two of six plasmids isolated contained the 2.5 kb DNA insert. The insert was excised from the gel and purified using a Millipore Ultrafree DA column (Millipore, Bedford, Mass.) and subsequently concentrated with an YM 100 filter (Millipore, Bedford, Mass.).

Cloning rNDST1 into pYES2/NTC yeast expression vector

The pYES2/NTC vector was digested with XhoI and XbaI restriction enzymes as described above. The linearized plasmid was purified using Millipore Ultrafree DA columns (Millipore, Bedford, Mass.) followed by concentration using YM 100 filters (Millipore, Bedford, Mass.). pYES2/NTC (Xhol/Xbal) (Invitrogen, Carlsbad, Calif.) and rNDST1 (Xhol/Xbal) linear DNA fragments were mixed at a 1:3 molar ratio (2 μl and 8 μl respectively) in 1×T4 ligase buffer (Promega Corp., Madison, Wis.) with 1 μl T4 ligase (Promega Corp., Madison, Wis.) and incubated at 4° C. overnight for ligation of the inserts into the vector. The following day, 4 μl of ligation mixture was mixed with One-shot E. coli TOP 10 E coli competent cells (Invitrogen, Carlsbad, Calif.). After a 1 hour incubation on ice, 45 seconds of heat shock at 42° C. and a 1 hour incubation in SOC medium at 37° C., 200 μl aliquots of the resultant cell suspensions were plated on LB plates supplemented with carbenicillin (75 μg/ml). The plates were incubated at room temperature overnight. Twenty-four colonies were isolated from the overnight plate and were grown in 5 ml LB cultures supplemented with 75 μg/ml carbenecillin. From these overnight cultures, plasmid DNA was purified using the plasmid DNA Miniprep protocol obtained from Qiagen (Chatsworth, Calif.). Plasmids were then screened for the insert DNA using Xhol and Xbal restriction enzymes as described herein. Further analyses were carried out using diagnostic restriction enzyme digestions. The restriction enzymes used for diagnostic steps included BamHI, EcoRI and SacI.

Competent Yeast Cells

S. cerevisiae cells were made competent using the S.c. EasyComp Transformation kit (Invitrogen, Carlsbad, Calif.). First, yeast cells (i.e., InvSc1 cells: Invitrogen, Carlsbad, Calif.) were streaked on a YPD (yeast extract, peptone, dextrose) agar plate and incubated for two days at 30° C. Single colonies were picked and grown in 10 ml of YPD media at 30° C. The resulting cultures were diluted to an OD₆₂₀ between 0.2 and 0.4 in a total volume of 10 ml YPD media. The cells were then grown at 30° C. with shaking until the OD₆₂₀ reached between 0.6 and 1.0 (between three and six hours). The cells were centrifuged at 500×g for 5 minutes at room temperature and the cell pellets were suspended in Solution I (Sc EasyComp Transformation Kit; Invitrogen, Carlsbad, Calif.). The cells were then centrifuged again at 500×g for 5 minutes, and the pellets were resuspended in Solution II (Sc EasyComp Transformation Kit; Invitrogen, Carlsbad, Calif.). The cells were then functionally “competent” at this stage. Two-hundred μl aliquots of competent yeast cells were frozen at −80° C.

Transformation of yeast cells with pYES2-rNDST1 plasmid

For transformation of the yeast cells with the pYES2-rNDST1 construct, 50 μl of competent cells were thawed and 2 μl of vector DNA was added, followed by the addition of Solution III (Sc EasyComp Transformation Kit; Invitrogen, Carlsbad, Calif.). The cells were mixed by vortexing vigorously, and the cell and DNA mixture was incubated for 1 hour at 30° C. to induce the uptake of DNA. The transformation reaction was mixed every 15 minutes by vortexing. The mixture was subsequently spread on plates containing selective medium and incubated at 30° C. for 2-4 days, until the transformed colonies appeared.

Expression of rNDST1 in Yeast Cells

The pYES2-rNDST1 construct was introduced into the INVSc1 yeast cell line. Transformed colonies, INVSc1/pYES2-rNDST1, were selected on glucose minus uracil agar plates. Single colonies were picked and inoculated in 5 ml CM-glucose minus uracil media (Teknova, Half Moon Bay, Calif.) and grown at 30° C. with shaking overnight. Overnight cultures were inoculated in CM-glucose minus uracil and grown until the OD₆₂₀ₙₘ was 0.5. At this stage, the cultures were harvested (3000×g for 5 minutes) and the pellets were resuspended in 90 ml of induction media, CM-galactose minus uracil media (Teknova, Half Moon Bay, Calif.). Five-ml aliquots were taken immediately before induction, centrifuged (1500×g at 4° C.) and washed with dH₂O (1/10 culture volume). The resulting pellet was stored at −80° C. until use. The remaining culture was grown overnight at 30° C. On the following day (approximately 16 hours), 45 ml of the culture (OD₆₂₀ₙₘ=1.12) was withdrawn, centrifuged and washed as described above, and then stored −80° C. The remaining cultures were allowed grow until 24 hours after induction (OD₆₂₀ₙₘ=1.2). These cultures were then harvested, washed and stored at −80° C. as described above.

Prepeparation of Yeast cell extracts:

Frozen yeast cell pellets were thawed and treated with Genotech Yeast-PE LB yeast protein extraction kit (St. Louis, Mo.). First, an equal volume of yeast suspension buffer (Genotech, St. Louis, Mo.) supplemented with P-mercaptoethanol was added to pellets. Then the yeast cell suspension was vortexed to obtain homogenous suspension, to which Longlife Zymolyase enzyme was added (Genotech, St. Louis, Mo.). The contents were mixed gently, followed by incubation at 37° C. for 1 hour. At the end of the incubation period, the suspension was centrifuged at 10,000×g for 5 minutes. Supernatants were discarded and the pellets were treated with 5-10 volumes of Yeast-PE LB containing 1 mM DTT and 1 mM PMSF. The suspensions were then vortexed and incubated on ice for 30 minutes, followed by a 1-2 minute incubation at 37° C. The resulting lysates were centrifuged at approximately 15,000×g in a microfuge for 1 hour at 4° C. The clear lysates were then used for assays and purification. Alternatively, yeast spheroplasts were broken up by vortexing with glass beads.

Monitoring NDST Activity

Expression of rNDST1 was monitored using a radioactive sulfotransferase assay with PAPS-³⁵S as sulfate donor and E. coli K5 polysaccharide or de-N-sulfated heparin as acceptor.

Purification of rNDST1 from Heparin-Sepharose CL-6B column

The heparin-sepharose CL-6B (Pharmacia LKB Biotechnology Inc., Piscataway, N.J.) was packed in a 1×10 cm column and washed extensively with dH₂O, then equilibrated with Buffer A (10 mM Tris pH 7.2, 20 mM MgCl₂, 2 mM CaCl₂, 10 mM β-mercaptoethanol, 0.1% Triton X-100 and 20% glycerol). Clear yeast extracts containing the rNDST1 enzyme were loaded onto the column, and the column was washed with the loading buffer until no protein was detected in the eluate. A linear gradient elution from 0.15 to 0.65 M NaCl was carried out in Buffer A. The eluted fractions were tested for rNDST1 activity using the radioactive sulfotransferase assay previously described. Fractions exhibiting high rNDST1 activity were pooled and concentrated using Apollo 7 concentrators (Orbital Biosciences, Topsfield, Mass.).

Radioactive sulfotransferase assay using E. coli K5 polysaccharide

Radioactive sulfotransferase assays were performed in either 10 mM Hepes buffer, pH 7.0 with 10 mM MnCl₂, 10 mM MgCl₂ and 5 mM CaCl₂, or MES buffer pH 6.5, with 10 mM MnCl₂. In each reaction, 10 μg acceptor sugar, 10 μM PAPS and 400,000-500,000 cpm ³⁵S-PAPS were incubated with 10-30 μl of cell lysate or 1-10 μl purified enzyme in 100 μl final volume at either 37° C. or room temperature for 1 hour, unless otherwise specified. The reactions were stopped by the addition of 10 μl of chondroitin sulfate (20 mg/ml) and 480 μl of 100% ethanol. The quenched reactions were then stored at −20° C. overnight to allow precipitation of the sugars. The following day, the tubes were centrifuged at approximately 10,000×g in an microfuge for 10 minutes. The supernatants were removed completely and the pellets were dissolved in 50 μl 10 mM TE buffer (pH 8.8, 0.1 M NaCl, 1 mM EDTA). G-25 Sephadex columns (Roche Biochemicals, Indianapolis, Ind.) were prepared by removing the excess storage buffer solutions by gravity flow, followed by centrifugation at 1100×g for 2 minutes at room temperature using a swinging bucket centrifuge. Then, 35 μl of resuspended quenched reaction solution was applied to the columns (Roche Biochemicals, Indianapolis, Ind.). The loaded columns were centrifuged at 1100×g for 2 minutes at room temperature. Flow-through fractions were collected and mixed with liquid scintillation fluid, followed by detection of radioactivity in a liquid scintillation counter.

Deacetylation and N-sulfation of E. coli K5 polysaccharide

Deacetylation of E. coli K5 or P. multicoda polysaccharides was carried out using rNDST1 enzyme in 50 mM MES buffer, pH 6.5, in the presence of 10 mm MnCl₂. N-sulfation was carried out in the same buffer where deacetylation occurred. To start N-sulfation, excess PAPS (200 μM-400 μM) was added and additional rNDST1 was added. The reactions were stopped either by heating the reaction mixture to 98° C. or simply storing them at −20° C. until analysis.

N-sulfation of Deacetylated Heparosan in PAPS cycle

Deacetylation of E. coli K5 polysaccharide was first performed as described above. Thirty μg of K5 polysaccharide was incubated in 50 mM MES buffer, pH 6.5, with 10 mM MnCl₂ and 20 μl of rNDST1 enzyme (with a specific activity of 176 pmol/min/mg) in 250 μl volume at room temperature overnight. The following day, 125 μl of the deacetylated reaction mixture was added to 125 μl of 50 mM MES, pH 6.5, 10 mM MnCl₂, 186 μM PAP, 100 mM PNPS, 0.5 μl dithiothreitol (DTT), 10 μl glutathione-S-transferase-arylsulfotransferase IV (GST-ASTIV). Additional rNDST1 was added 1-2 minutes after the PAPS cycle was initiated. The reactions were allowed to proceed overnight and products were stored at −20° C. until analysis.

High-performance liquid chromatography (HPLC) Analysis of N-Deacetylated disaccharides

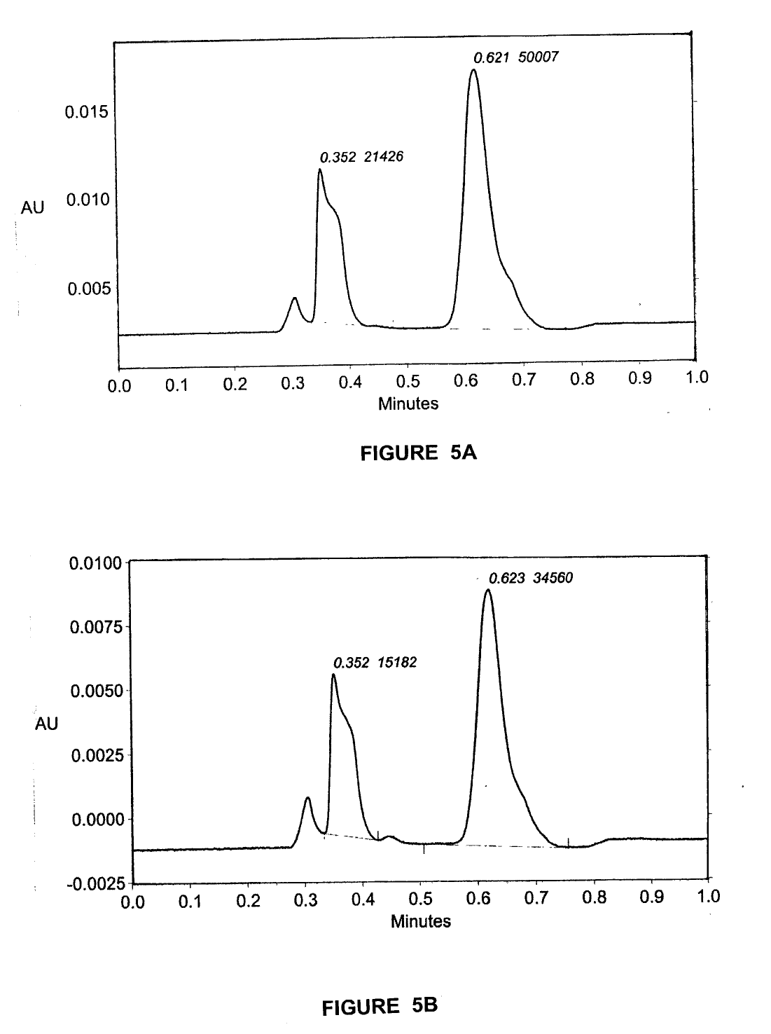

Reaction mixture were first dialyzed against water using Millipore “V’ series membranes, unless the sample volume was higher than 100 μl, in which case the sample was concentrated by evaporation. The polysaccharides were digested with combination of three enzymes—Heparinase I, Heparinase II (Sigma Chemical Company, St. Louis, Mo.) and Heparitinase I (Seikagaku, Falmouth, Mass.)—at 30° C. for two hours, followed by overnight incubation at 37° C. The following day, the proteins were denatured by boiling and pelleted by centrifugation. The clear supernatants were saved. Samples were subsequently analyzed on reverse-phase HPLC. Retention time for uronic acid (UA)-GlcV was 0.35 min and for UA-GlcNac was 0.62 min, determined using a UV detector to monitor unsaturated UA at 232 nm (FIGS. 5a, 5b).

Ion-Exchange Chromatography—High-Performance Liquid Chromatography (IEC-HPLC) Analysis of N-Sulfated Disaccharides

Supernatants obtained above were treated with anthranilamide (2-AB) and NaBH₄CN (to derivatize the disaccharides) at 65° C. for 2 hours. 2-AB derivatized disaccharides were cleaned using acetonitrile, followed by dissolution in dH₂O. Finally, the samples were analyzed using IEC-HPLC. The fluorescent tags were monitored at 330 nm excitation wavelength and 420 nm emission wavelength. Retention time for UA-GlcNAc was 8.4 min and for UA-GlcNS was 16.5 min.

The results of the experiments presented herein are now described.

Cloning rNDST1 gene in yeast expression system

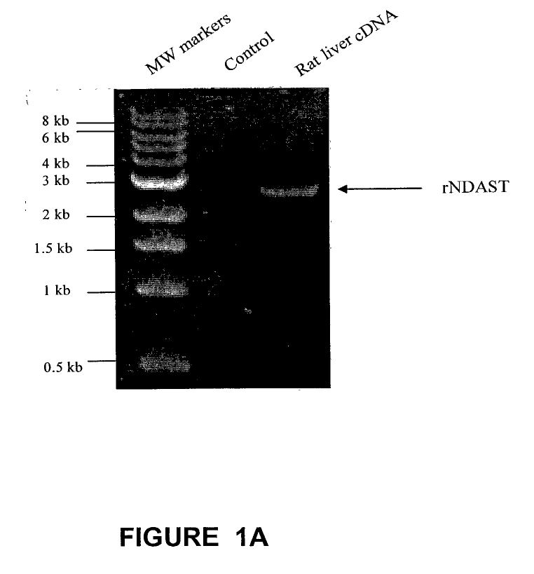

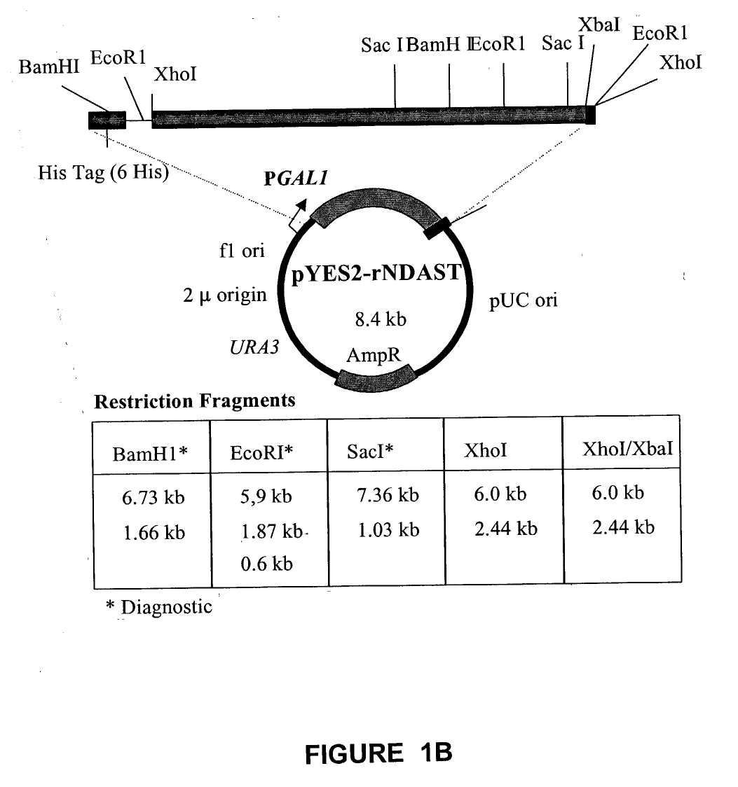

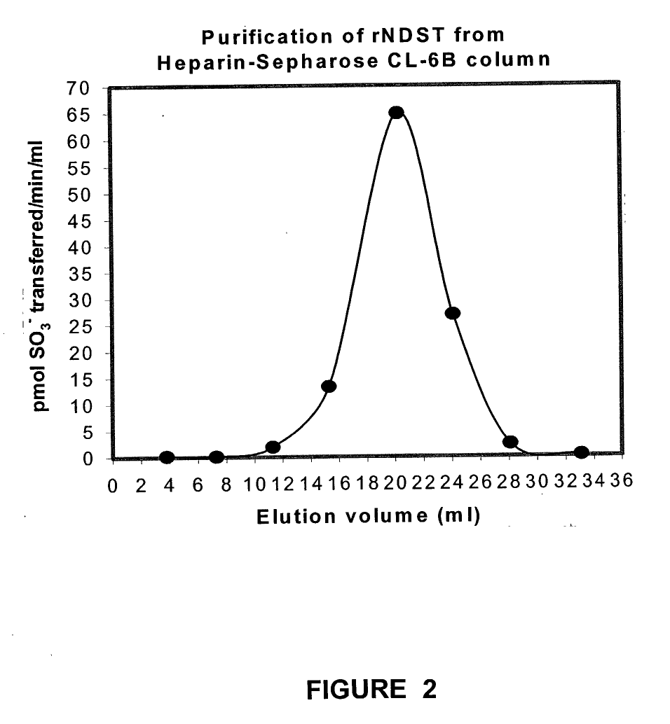

A single 2.5 bp DNA band corresponding to a truncated rNDST1 gene was obtained after PCR amplification. (FIG. 1a). This fragment was first cloned into the pCR-Blunt vector, then subcloned into the pYES2/NTC vector to obtain the 8.4 kb final construct (FIG. 1b). Restriction mapping indicated that the cloned gene was identical to the rat liver heparan sulfate/heparin N-deacetylase/N-sulfotransferase originally cloned by Hirschberg (U.S. Pat. No. 5,541,095).

Expression of rNDST1 in yeast cells

The expression of the rNDST1 enzyme was achieved in yeast cells by galactose induction of the gene for the rNDST1 enzyme, which was under the control of GAL1 promoter. Transcription from the GAL1 promoter is repressed with glucose and is induced by removal of glucose and addition of galactose (Invitrogen Yeast Expression Manual, Carlsbad, Calif.). The NDST activity reached maximum levels after 24 hours of incubation in galactose-containing media. In Table 1, expression of rNDST1 is shown as pmol SO₃⁻ transferred to K5 polysaccharide. At the beginning of rNDST1 expression, there was very little rNDST1 activity in the yeast cell lysate. However, the activity increased over time and reached maximum levels after 24 hours after induction with galactose. As illustrated in Table 1, a yeast culture activity of 5850 pmol/min/L of yeast culture was achieved in this particular instance. The highest rNDST1 activity obtained was 11240 pmol/min/L yeast culture after a 24 hour induction period with galactose.

TABLE 1
Expression of rNDST1 in InvSc1/pYES2-rNDST1
Radioactive sulfotransferase assays were conducted at
pH 7.0 using 10 μl of yeast cell lysates (as described
below under “Yeast cell lysis”) obtained from the
yeast pellets (harvested at indicated induction times)
and K5 polysaccharide. Details of the assay are as
described above in the Materials and Methods section.

| Induction Time | pmol/min/L yeast culture |
|---|---|
| 0 hr | 5.5 |
| 16 hr | 4200 |
| 24 hr | 5850 |

Yeast cell lysis

Yeast cells can be lysed using an combination of an enzyme, such as Zymolyase® (Genotech, St. Louis, Mo.), followed by detergent (Genotech, St. Louis, Mo.) or by mechanical means of disruption (e.g., using glass beads). Zymolyase digests the cell wall of yeast cells, converting the cells into spheroplasts. The spheroplasts are then easily broken with detergent treatment or mechanical disruption. When the InvSc1/pYES2-rNDST1 cells were lysed using the above method, the highest amount of intracellular yeast proteins were released using the Genotech method based on SDS-PAGE (Data not shown). The clear cell lysates were used in radioactive sulfotransferase assays without any artifactual interference.

Acceptor specificity of rNDST1

The rNDST1 activity was assayed in the presence of different sugar acceptors using the yeast cell lysate obtained from InvSc1/pYES2-rNDST1 cultures. When E. coli K5 polysaccharide was used as acceptor, the sulfotransferase activity measured was a product of both the N-deacetylation and the N-sulfation activities. This activity was 196 pmol/min/ml cell lysate at pH 7.0. The enzyme had a slightly lower activity (159 pmol/min/ml cell lysate) when DNSH (de-N-sulfated heparin) was used as an acceptor. DNSH is a mixture containing primarily GlCN with some O-sulfated GlcNAc residues, and the observed rNDST1 activity using DNSH arose primarily as a result of N-sulfotransferase activity (there is a minor contribution of some combined N-deacetylase and N-sulfotransferase enzymatic activities). If the acceptor CDSNAcHS (completely desulfated N-acetylated Heparan sulfate), which resembles K5 polysaccharide with a number of iduronic residues, was used, NDST activity was found to be 168 pmol/min/ml cell lysate. Incidentally, this activity was comparable to that obtained using K5 as an acceptor. However, rNDST1 exhibited low enzymatic activity with NDSNAcH (N-desulfated N-acetylated Heparin) as an acceptor. This is likely due to the fact that the O-sulfations inhibited deacetylation activity of the enzyme. Chondroitin was used as negative control as it is not a substrate for rNDST1 due to the presence of GalNAc instead of GlcNAc in the repeating disaccharide units.

TABLE 2
rNDST1 activities at pH 7.0 with different sugar acceptors.
Experiments were conducted using 10 μl of yeast cell lysate
(INVSc1/pYES2-rNDST1) obtained with Genotech yeast lysis method.

| Acceptor | pmol/min/ml cell lysate |
|---|---|
| K5 polysaccharide | 196 |
| D-NSHeparin | 159 |
| NDSNAc-Heparin | 20 |
| CDSNAc-Heparan Sulfate | 168 |
| No Acceptor | 1.6 |
| Chondroitin | 2.5 |

Purification of rNDST1:

Because NDST has an affinity to heparin-sepharose, the purification of the enzyme was facilitated using affinity chromatography. The enzyme bound to the heparin sepharose column adequately, and no sulfotransferase activity was detectable in wash and flow through fractions. At the end of the gradient elution, the fractions having high sulfotransferase activity were saved, where the highest activity fractions were pooled separately from the lower activity fractions. The sulfotransferase activities were monitored at pH 6.5 in the presence of 10 mM Mn²⁺ (assay as described above). In one particular batch purification, 48% of the activity was recovered from the heparin-sepharose CL-6B column after a linear NaCl gradient elution. SDS-PAGE analysis did not demonstrate a significant protein band corresponding rNDST1, suggesting that expressed enzyme is very active with respect to both activities. The purified enzyme can be stored in elution buffer containing glycerol at −20° C. without a significant decrease in either activity. However, prolonged storage of the cell lysate after preparation with Genotech Zymolyase+detergent treatment led to an inactive enzyme. If the detergent treatment was substituted with glass beads, the enzyme remained active during prolonged storage.

New sulfotransferase assay for rNDST1

Because the N-deacetylation and N-sulfation reactions require different conditions for their optimum activities (Wei and Swiedler, 1999, J. Biol. Chem. 274:1966-1970), investigators previously used separate assays for N-deacetylation and N-sulfation reactions catalyzed by the single NDST enzyme (Aikawa et al., 2001, J. Biol. Chem. 276:5876-5882). While N-deacetylase activity is usually monitored using ³H-labeled K5 (³H-N-acetyl heparosan) as substrate at pH 6.5 (MES buffer) in the presence of Mn²⁺, N-sulfation activity is monitored using chemically deacetylated K5 polysaccharide and PAPS-³⁵S at pH 7.0 (Hepes) in the presence of 10 mM Mg²⁺, 5 mM Ca²⁺ and 10 mM Mn²⁺ ( Aikawa et al., 2001, J. Biol. Chem. 276:5876-5882).

N-deacetylation and N-sulfation are coupled in vivo, and N-deacetylation is prerequisite for N-sulfation (Kushe-Gullberg et al.,1998, J. Biol. Chem. 273:11902-11907). These conditions require 50 mM MES buffer at pH 6.5 and 10 mM MnCl₂ In order to produce efficient N-sulfated polysaccharides from GlcNAc-GlcA repeating polysaccharides such as K5, the modified sulfotransferase activity that measures the coupling activities of SO₃⁻ transfer to carbohydrate following N-deacetylation was considered to be more suitable not just for monitoring the activities, but also for manufacturing the N-sulfated sugars. The reaction requires Mn²⁺ ions and elimination of the Mn²⁺ in the sulfotransferase assay did not result in any measurable rNDST1 activity. The buffer system commonly used to measure sulfotransferase activities (HEPES pH 7.0 with Mn²⁺, Mg²⁺, Ca²⁺) (Aikawa et al., 2001, J. Biol. Chem. 276:5876-5882) may not reflect optimal conditions for monitoring both activities, since rNDST1 activity in this buffer is seven times lower than that in MES pH 6.5 with Mn²⁺. Addition of non-stoichiometric amounts of the chelating agent EDTA increased the modified sulfotransferase activity 3-fold, suggesting that Mg²⁺ and Ca²⁺most likely inhibit the combined N-deacetylase/N-sulfotransferase activity (see Table 3).

TABLE 3
Effect of buffer systems on observed activities of rNDST1
with K5 polysaccharide as acceptor. The reactions were
carried out with 3 μl of purified rNDST1 in a radioactive
sulfotransferase assay.

| Buffer system | pmol/min/ml enzyme |
|---|---|
| Hepes, pH 7.0, 10 mM MnCl₂, MgCl₂. | 218.7 |
| 5 mM CaCl₂ | |
| Hepes, pH 7.0, 10 mM MnCl₂, MgCl₂. | 633.4 |
| 5 mM CaCl₂ + 5 mM EDTA | |
| MES pH 6.5, 10 mM MnCl₂, | 1550.1 |
| MES pH 6.5 | 1.5 |
| Control (no enzyme) | 2.4 |

N-deacetylation kinetics

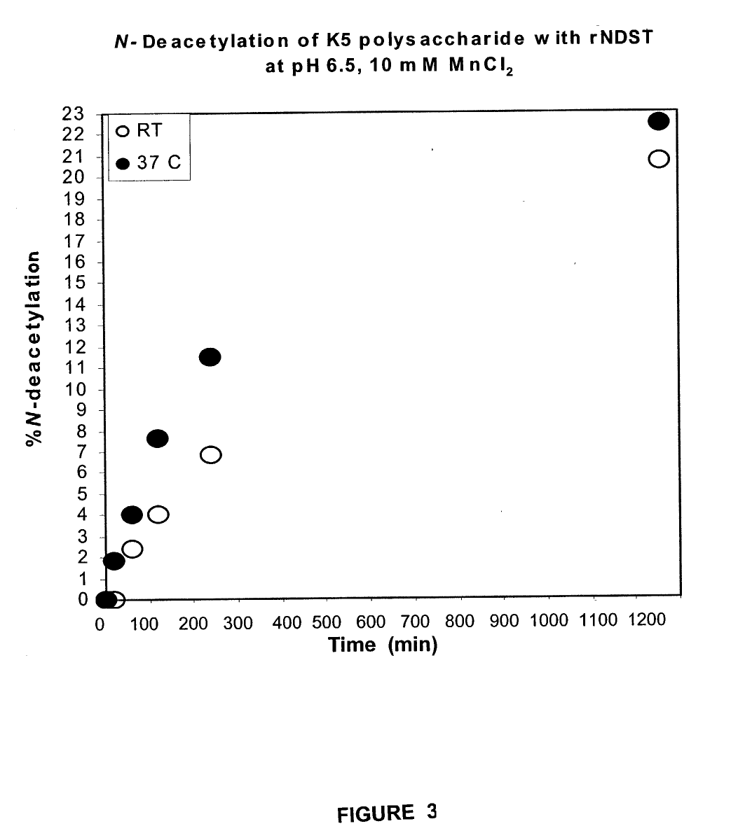

N-deacetylation reactions were followed by the formation of product, GlcN (deacetylated N-acetyl glucosamine) (FIGS. 5a, 5b). E. coli K5 polysaccharide was incubated with rNDST1 in the presence of 10 mM MnCl₂ at pH 6.5, at room temperature and 37° C. The reactions were stopped by incubating the reaction mixture at 98° C. for 2-3 minutes. The N-deacetylation rate was analyzed by HPLC after enzymatic digestion as described in the Materials and Methods section. For analysis of the activity, the extent of GlcN formation (% product) was plotted against time (FIG. 3). There is a two fold difference in deacetylation between room temperature and 37° C. Both reactions reached saturation at 20-23% deacetylation levels when incubated overnight.

N-sulfation kinetics of rNDST1

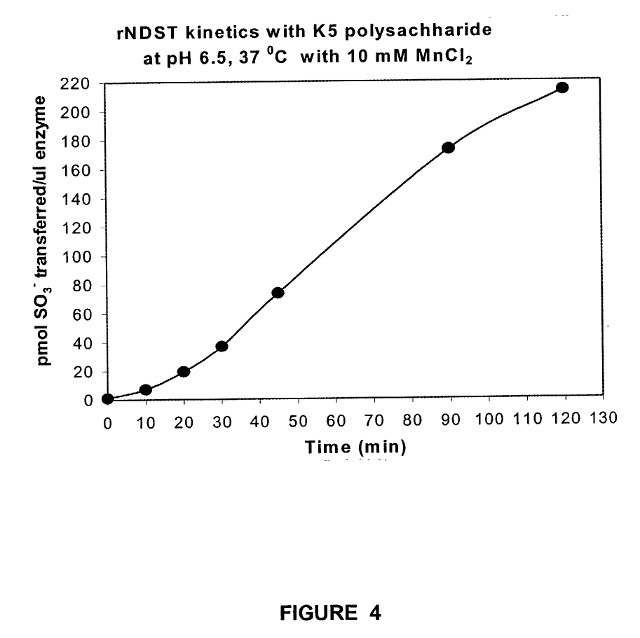

N-sulfation of K5 polysaccharide with rNDST1 was monitored over time. The activity was measured as a function of sulfate transfer to K5 polysaccharide. The kinetic data were plotted as a sigmoidal curve as a result of combined N-deacetylase/N-sulfotransferase activity. Because there was little GlcN available at the beginning of the assay, N-sulfotransferase activity is slow. As more GlCN became available, N-sulfotransferase activity increased. The N-sulfotransferase activity was linear for about 60 minutes between 20 to 90 minutes into the assay (FIG. 4).

Production of N-sulfated polysaccharides for Heparin Biosynthesis: N-acetylation and N-sulfation of E. coli K5 and P. multicoda polysaccharides

P. multicoda has a capsular polysaccharide (PM PS) that contains a GlcNAc-GlcA repeating unit, identical to the E. coli K5 polysaccharide (DeAngelis and White, 2002, J. Biol. Chem. 277:7209-7213). PM PS was tested in N-deacetylation and N-sulfation experiments using rNDST1. Radioactive sulfotransferase experiments have demonstrated that sulfate transfer occurs from PAPS to PM PS polysaccharide with rNDST1.

N-sulfations were carried out directly on E. coli polysaccharide K5 or P. multicoda polysaccharide using rNDST1 and PAPS. The extent of N-sulfation was determined based on HPLC separation of derivatized disaccharides obtained after heparinase I and II, and heparitinase digestions.

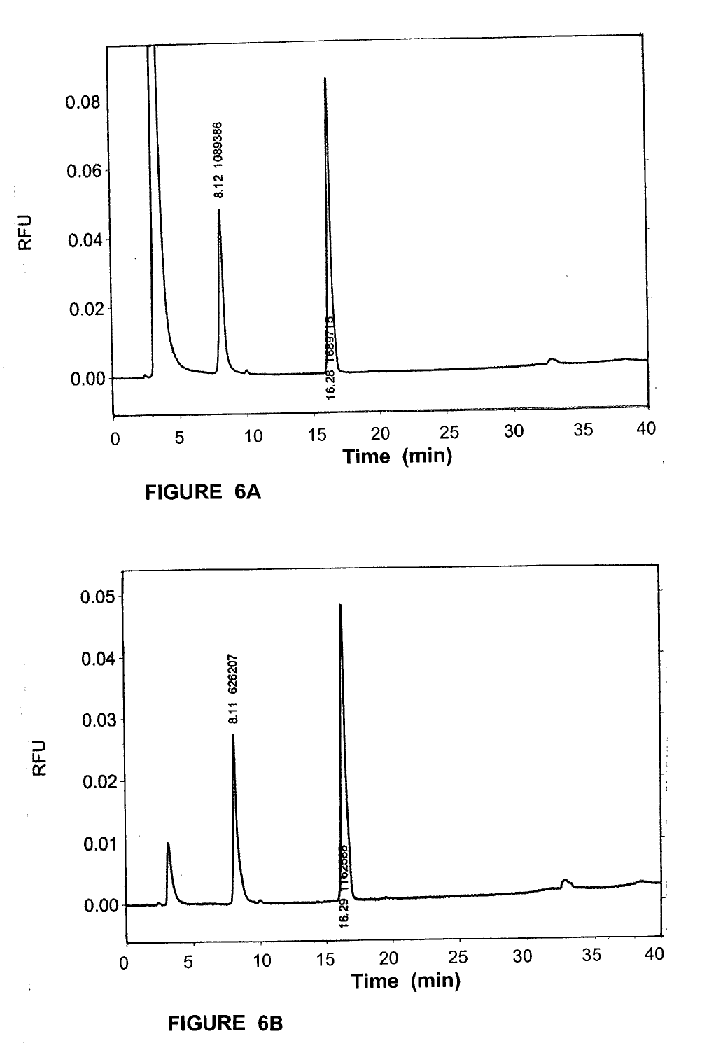

After overnight incubation at 35° C. in the presence of 100 μM PAPS and 10 μl rNDST1 enzyme (231 pmol/min/mg), 25% of the PM PS was N-sulfated. The reaction was continued by the addition of more PAPS (400 μM) and 5 μl additional rNDST1. After two days, 56% of the PM PS was found to be N-sulfated. If the N-deacetylation was carried out at 35° C. overnight first with 10 μl rNDST1 enzyme (by omitting PAPS from the reaction), 31% of the GlcNAc was found to be deacetylated (FIG. 5B), and following overnight incubation with 400 μM PAPS and 5 μl rNDST1, the reaction yielded 65% N-sulfated PM PS (FIG. 6B).

Similarly, overnight incubation at 35° C. in the presence of 100 μM PAPS and 10 μl rNDST1 enzyme, 18% of the E. coli K5 polysaccharide was found to be N-sulfated. The same reaction, continued overnight by addition of more PAPS (400 μM) and 5 μl more rNDST1 (231 pmol/min/mg), yielded 50% N-sulfated E. coli K5 polysaccharide. If only N-deacetylation was first carried out at 35° C. overnight with 10 μl of enzyme (231 pmol/min/mg) (FIG. 5A), 30% of the GlcNAc was found to be N-deacetylated. Following overnight incubation with 400 μM PAPS and 5 μl rNDST1 in the same reaction, 60% N-sulfated E. coli K5 polysaccharide was obtained (Table 4, FIG. 6A).

N-sulfation in PAPS cycle

N-sulfated heparosan can be synthesized using the PAPS cycle where PAPS is being produced transiently (U.S. Pat. No. 6,255,088). First, N-deacetylation was carried out separate from the PAPS cycle using rNDST1 at pH 6.5, 10 mM MnCl₂. In the cycle, N-deacetylated K5 polysaccharide was mixed directly with the cycle components (transient production of PAPS from PAP via PNPS and ASTIV (Arylsulfotrasnferase IV)). The PAPS cycle was performed at pH 6.5 with 10 mM MnCl₂ where the rNDST1 was found to be more active. After N-deacetylation at room temperature, N-sulfation in the PAPS cycle yielded 24% N-sulfated K5 polysaccharide. If native K5 polysaccharide was included in the cycle, a low yield of N-sulfated polysaccharide (4-5%) was obtained due to inhibitory effects of the components included in the PAPS cycle or N-deacetylase activity. Further, if N-deacetylation is carried out at 37° C. overnight and the resulting reaction mixture combined with PAPS cycle components, the resultant N-sulfation yield reaches 38% N-sulfated K5 polysaccharide.

TABLE 4
N-sulfation of bacterial polysaceharides containing
GlcNAc-GlcA repeating units. Percent conversions are shown
from the HPLC profile (See Figures 5 and 6). The amount of product
was estimated based on 10 μg starting material and percent
conversion, assuming that analysis showed all the possible
N-sulfations (either E. coli K5 or PM polysaccharide).
O/N: overnight, DA: N-deacetylation, NS: N-sulfation. rNDST1
(231 pmol/min/mg).

| | | | | % N-Sulfation |
|---|---|---|---|---|
| Acceptor | Reaction (Time) | PAPS | rNDST1 | (Amount) |
| E.coli K5 | NS (O/N) | 100 μM | 10 μl | 18 (1.6 μg) |
| E.coli K5 | NS (2 O/N) | 500 μM | 15 μl | 50 (5 μg) |
| E.coli K5 | DA/NS (2 O/N) | 400 μM | 15 μl | 60 (6 μg) |
| PM PS | NS (O/N) | 100 μM | 10 μl | 25 (2.25 μg) |
| PM PS | NS (2 O/N) | 500 μM | 15 μl | 55 (5.56 μg) |
| PM PS | DA/NS (2 O/N) | 400 μM | 15 μl | 65 (6.5 μg) |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid comprising SEQ ID NO:3, and homologs, variants, mutants and fragments thereof, encoding a bifunctional enzyme having both N-deacetylase and N-sulfotransferase activity, wherein both activities are active in the same in vitro reaction mixture.

2. An isolated nucleic acid encoding a bifunctional enzyme having both N-deacetylase and N-sulfotransferase activity, wherein both activities are active in the same in vitro reaction mixture, said isolated nucleic acid sharing greater than 99% sequence identity with the nucleic acid of SEQ ID NO:3.

3. An isolated nucleic acid comprising SEQ ID NO:3 encoding a bifunctional enzyme having both N-deacetylase and N-sulfotransferase activity, wherein both activities are active in the same in vitro reaction mixture.

4. An isolated polypeptide comprising SEQ ID NO:4, and homologs, variants, mutants and fragments thereof, having both N-deacetylase and N-sulfotransferase activity, wherein both activities are active in the same in vitro reaction mixture.

5. An isolated polypeptide having both N-deacetylase and N-sulfotransferase activity, wherein both activities are active in the same in vitro reaction mixture, said isolated polypeptide sharing greater than 99% sequence identity with the polypeptide of SEQ ID NO:4.

6. An isolated polypeptide comprising SEQ ID NO:4 having both N-deacetylase and N-sulfotransferase activity, wherein both activities are active in the same in vitro reaction mixture.

7. The isolated polypeptide of claim 6, further comprising a poly-histidine sequence.

8. The isolated polypeptide of claim 7, further comprising an Xpress™ epitope.

9. The isolated polypeptide of claim 8, further comprising an enterokinase cleavage site.

10. The isolated polypeptide of claim 9, wherein the enterokinase cleavage site is immediately adjacent to SEQ ID NO:4 on the N-terminal side of SEQ ID NO:4.

11. The isolated polypeptide of claim 10, wherein the poly-histidine sequence, the Xpress™ epitope, and the enterokinase cleavage site have been removed using an enterokinase.

12. A method of N-deacetylating and N-sulfating a saccharide, the method comprising contacting a saccharide with a composition comprising a polypeptide encoded by the nucleic acid of SEQ ID NO:3, or homologs, variants, mutants and fragments thereof, under conditions sufficient to support both activities in the same reaction mixture, such that the saccharide is modified by N-deacetylation and N-sulfation reactions catalyzed by the polypeptide.

13. A method of N-deacetylating and N-sulfating a saccharide, the method comprising contacting a saccharide with a composition comprising a polypeptide under conditions sufficient to support both activities in the same reaction mixture, said isolated polypeptide sharing greater than 99% sequence identity with the polypeptide encoded by the nucleic acid set forth in SEQ ID NO:3, such that the saccharide is modified by N-deacetylation and N-sulfation reactions catalyzed by the isolated polypeptide.

14. A method of N-deacetylating and N-sulfating a saccharide, the method comprising contacting a saccharide with a composition comprising a polypeptide encoded by the nucleic acid of SEQ ID NO:3, under conditions sufficient to support both activities in the same reaction mixture, such that the saccharide is modified by N-deacetylation and N-sulfation reactions catalyzed by the polypeptide.

15. A method of N-deacetylating and N-sulfating a saccharide, the method comprising contacting a saecharide with a composition comprising the polypeptide of claim 9, under conditions sufficient to support both activities in the same reaction mixture, such that the saccharide is modified by N-deacetylation and N-sulfation reactions catalyzed by the polypeptide.

16. A method of N-deacetylating and N-sulfating a saccharide, the method comprising contacting a saccharide with a composition comprising the polypeptide of claim 10, under conditions sufficient to support both activities in the same reaction mixture, such that the saccharide is modified by N-deacetylation and N-sulfation reactions catalyzed by the polypeptide.

17. The method of claim 16, wherein the N-deacetylation and N-sulfation reactions are both conducted in the same reaction mixture.

18. The method of claim 17, wherein the composition comprising the polypeptide is a cell extract.

19. The isolated nucleic acid of claim 3, said nucleic acid further comprising a nucleic acid specifying a promoter/regulatory sequence operably linked thereto.

20. The isolated nucleic acid of claim 19, wherein the promoter is functional in a yeast or fungus cell expression system.

21. The isolated nucleic acid of claim 19, wherein the promoter is functional in a bacterial cell expression system.

22. The isolated nucleic acid of claim 19, wherein the promoter is functional in an insect cell expression system.

23. The isolated nucleic acid of claim 19, wherein the promoter is functional in a mammalian cell expression system.

24. A vector comprising an isolated nucleic acid comprising SEQ ID NO:3.

25. The vector of claim 24, the vector further comprising a nucleic acid specifying a promoter/regulatory sequence operably linked to the isolated nucleic acid.

26. The vector of claim 25, wherein the isolated nucleic acid is expressed when introduced into a cell.

27. The vector of claim 26, wherein the isolated nucleic acid further comprises at the 5′ end a nucleic acid comprising at least one of:
a) a six-histidine sequence to aid in purification of the expressed polypeptide;
b) an Xpress™ epitope to aid in detection of the polypeptide; and
c) an enterokinase recognition site for subsequent cleavage from the polypeptide of the purification and detection sequences set forth in a) and b) above.

28. A recombinant cell comprising an isolated nucleic acid comprising SEQ ID NO:3.

29. A recombinant cell comprising the vector of claim 24.

30. A recombinant cell comprising the vector of claim 25.

31. A recombinant cell comprising the vector of claim 26.

32. A recombinant cell comprising the vector of claim 27.

33. A method of detecting sulfotransferase activity in an assay mixture of an isolated polypeptide encoded by a nucleic acid comprising SEQ ID NO:3, wherein the assay mixture comprises:
a) a buffer with sufficient buffering capacity at pH 7.0;
b) MnCl₂;
c) MgCl₂;
d) CaCl₂;
e) an acceptor sugar;
f) ³⁵S-PAPS; and
g) non-labeled PAPS.

34. A method of detecting sulfotransferase activity in an assay mixture of an isolated polypeptide encoded by a nucleic acid comprising SEQ ID NO:3, wherein the assay mixture comprises:
a) a buffer with sufficient buffering capacity at pH 7.0;
b) 10 mM MnCl₂;
c) 10 mM MgCl₂;
d) 5 mM CaCl₂;
e) 10 μg of an acceptor sugar;
f) between 400,000 cpm and 500,000 cpm ³⁵S-PAPS; and
g) 10 μM non-labeled PAPS.

35. A method of detecting sulfotransferase activity in an assay mixture of an isolated polypeptide encoded by a nucleic acid comprising SEQ ID NO:3, wherein the assay mixture comprises:
a) a buffer with sufficient buffering capacity at pH 6.5;
b) MnCl₂;
c) MgCl₂;
d) CaCl₂;
e) an acceptor sugar;
f) ³⁵S-PAPS; and
g) non-labeled PAPS.

36. A method of detecting sulfotransferase activity in an assay mixture of an isolated polypeptide encoded by a nucleic acid comprising SEQ ID NO:3, wherein the assay mixture comprises:
a) a buffer with sufficient buffering capacity at pH 6.5;
b) 10 mM MnCl₂;
c) 10 mM MgCl₂;
d) 5 mM CaCl₂;
e) 10 μg of an acceptor sugar;
f) between 400,000 cpm and 500,000 cpm ³⁵S-PAPS; and
g) 10 μM non-labeled PAPS.

37. A method of detecting sulfotransferase activity in an assay mixture of an isolated polypeptide encoded by a nucleic acid comprising SEQ ID NO:3, wherein the steps of the method comprise:
a) preparing the assay mixture of one of claims 33, 34, 35 or 36;
b) adding to the assay mixture an isolated polypeptide encoded by a nucleic acid comprising SEQ ID NO:3;
c) allowing the reaction to proceed;
d) isolating the sugars from the reaction; and
e) measuring the amount of ³⁵S present on the isolated sugars.

38. The method of claim 37, wherein the sugar acceptor is chosen from the group consisting of:
a) E. coli K5 polysaccharide;
b) de-N-sulfated heparin;
c) N-desulfated N-acetylated heparin; and
d) completely desulfated N-acetylated heparan sulfate.

39. A method of detecting sulfotransferase activity in an assay mixture of an isolated polypeptide encoded by a nucleic acid comprising SEQ ID NO:3, the method comprising preparing an assay mixture, adding to the assay mixture an isolated polypeptide encoded by a nucleic acid comprising SEQ ID NO:3, allowing the reaction to proceed, isolating the sugars from the reaction; and measuring the amount of ³⁵S present on the isolated sugars, wherein the buffer system used in the assay mixture is selected from the group consisting of:
a) pH 7.0, 10 mM MnCl₂, 10 mM MgCl₂, 5 mM CaCl₂;
b) pH 7.0, 10 mM MnCl₂, 10 mM MgCl₂, 5 mM CaCl₂, 5 mM EDTA; and
c) pH 6.5, 10 mM MnCl₂.
